(19) United States
(12) Patent Application Publication
(10) Pub. No.: US 2017/0290617 A1
(43) Pub. Date: Oct. 12, 2017

(54) FORCE SENSING CATHETER WITH A MAGNET AND AN INDUCTIVE SENSOR

(71) Applicant: Boston Scientific Scimed Inc., Maple Grove, MN (US)

(72) Inventors: Darrell L. Rankin, Milpitas, CA (US); John C. Potosky, Gamerville, NV (US)

(21) Appl. No.: 15/471,220

(22) Filed: Mar. 28, 2017

Related U.S. Application Data
(60) Provisional application No. 62/320,289, filed on Apr. 8, 2016.

(51) Int. Cl. A61B 18/00

(52) U.S. Cl. CPC A61B 18/00; A61B 2018/00577; A61B 2018/00964; A61B 2018/00059; A61B 2018/00351

(57) ABSTRACT

A catheter adapted to measure a contact force includes a proximal segment, a distal segment, a spring segment extending from the proximal segment to the distal segment, at least one magnet disposed on one of the proximal segment and the distal segment, and at least one inductive sensor disposed on the other one of the proximal segment and the distal segment opposite the at least one magnet. The spring segment is configured to permit displacement between the distal segment and the proximal segment in response to an application of the force on the distal segment. The at least one inductive sensor includes a first plate of high magnetic permeability material, and at least one coil disposed adjacent to the first plate of high magnetic permeability material. The coil is configured to output a signal indicative of the displacement between the inductive sensor and the opposite magnet.

### CROSS-REFERENCE TO RELATED APPLICATION

This application claims priority to Provisional Application No. 62/320,289, filed Apr. 8, 2016, which is herein incorporated by reference in its entirety.

### TECHNICAL FIELD

The present disclosure relates generally to various force sensing catheter features.

### BACKGROUND

In ablation therapy, it may be useful to assess the contact between the ablation element and the tissue targeted for ablation. In interventional cardiac electrophysiology (EP) procedures, for example, the contact can be used to assess the effectiveness of the ablation therapy being delivered. Other catheter-based therapies and diagnostics can be aided by knowing whether a part of the catheter contacts targeted tissue, and to what degree the part of the catheter presses on the targeted tissue. The tissue exerts a force back on the catheter, which can be measured to assess the contact and the degree to which the catheter presses on the targeted tissue.

The present disclosure concerns, amongst other things, systems for measuring a force with a catheter.

### SUMMARY

The present disclosure relates to devices, systems, and methods for measuring a contact force experienced by a catheter tip.

Example 1 is a catheter adapted to measure a contact force. The catheter includes a proximal segment, a distal segment, a spring segment extending from the proximal segment to the distal segment, at least one magnet disposed on one of the proximal segment and the distal segment, and at least one inductive sensor disposed on the other one of the proximal segment and the distal segment opposite the at least one magnet. The spring segment is configured to permit displacement between the distal segment and the proximal segment in response to an application of the force on the distal segment. The at least one inductive sensor includes a first plate of high magnetic permeability material, and at least one coil disposed adjacent to the first plate of high magnetic permeability material. The coil is configured to output a signal indicative of the displacement between the inductive sensor and the opposite magnet.

In Example 2, the catheter of Example 1, wherein edges of the first plate of high magnetic permeability material extend beyond edges of the at least one coil.

In Example 3, the catheter of Example 1, wherein the at least one inductive sensor further includes a second plate of high magnetic permeability material, wherein the at least one coil is disposed between the first plate of high magnetic permeability material and the second plate of high magnetic permeability material.

In Example 4, the catheter of Example 3, wherein edges of the first plate of high magnetic permeability material and edges of the second plate of high magnetic permeability material extend beyond edges of the at least one coil.

In Example 5, the catheter of any of Examples 1-4, wherein the at least one coil includes a plurality of axially spaced coils.

In Example 6, the catheter of any of Examples 1-5, wherein the at least one coil is a flat coil of one or more flexible printed circuit conductive layers.

In Example 7, the catheter of any of Examples 1-6, wherein the at least one magnet is disposed on the distal segment, and the at least one inductive sensor is disposed on the proximal segment.

In Example 8, the catheter of any of Examples 1-7, wherein the distal segment includes an ablation element configured to deliver ablation therapy.

In Example 9, the catheter of any of Examples 1-8, wherein at least one magnet includes a plurality of magnets and the at least one inductive sensor includes a plurality of inductive sensors.

In Example 10, the catheter of Example 9, wherein the plurality of magnets consists of three magnets and the plurality of inductive sensor consists of three inductive sensors circumferentially arrayed evenly about a longitudinal axis.

In Example 11, the catheter of any of Examples 1-10, wherein the signal indicative of the displacement between the inductive sensor and the opposite magnet is a change in an alternating voltage amplitude resulting from changes in a magnetic saturation of the first plate of high magnetic permeability material caused by changes in a distance between the inductive sensor and the opposite magnet.

Example 12 is a system adapted to measure a catheter contact force. The system includes a catheter according to any of Examples 1-11 and control circuitry configured to receive, for each of the plurality of inductive sensors, the signal indicative of the displacement between the magnet and the inductive sensor, and calculate at least one of a magnitude and a direction of the contact force based at least in part on the received signals.

In Example 13, the system of Example 12, wherein the spring segment includes an elastic element connecting the proximal segment to the distal segment to permit displacement between the distal segment and the proximal segment in response to an application of the force on the distal segment, wherein the control circuitry is further configured to calculate the at least one of the magnitude and the direction of the contact force based at least in part on a spring constant for the elastic element.

In Example 14, the system of either of Examples 12 or 13, wherein the control circuitry is further configured to deliver an alternating sinusoidal electrical current to the at least one coil of each of the plurality of inductive sensors to produce an alternating voltage across the at least one coil.

Example 15 is a method of determining a contact force exerted on a catheter having an elastic element disposed between a proximal segment having a plurality of coils adjacent to at least one plate of high magnetic permeability material, and a distal segment having a plurality of magnets opposite the coils. The method includes delivering an alternating sinusoidal electrical current to the plurality of coils to produce an alternating voltage across each of the plurality of coils; measuring an amplitude of the alternating voltage produced across each of the plurality of coils, wherein for at least one of the plurality of coils, the amplitude of the alternating voltage decreases as the magnet opposite the coil moves closer to the coil and reduces an effective magnetic permeability of the at least one plate of high magnetic permeability material as the contact force is exerted on the catheter; and calculating at least one of the magnitude and the direction of the contact force based on the measured amplitude of the alternating voltage produced across each of the plurality of coils and on a spring constant for the elastic element.

Example 16 is a catheter adapted to measure a contact force. The catheter includes a proximal segment, a distal segment, a spring segment extending from the proximal segment to the distal segment, a plurality of magnets disposed on one of the proximal segment and the distal segment, and a plurality of inductive sensors disposed on the other one of the proximal segment and the distal segment. Each of the plurality of inductive sensors is opposite a different one of the plurality of magnets. The spring segment is configured to permit displacement between the distal segment and the proximal segment in response to an application of the force on the distal segment. Each of the plurality of inductive sensors includes a first plate of high magnetic permeability material and at least one coil disposed adjacent to the first plate of high magnetic permeability material. The coil is configured to output a signal indicative of the displacement between the inductive sensor and the corresponding opposite magnet.

In Example 17, the catheter of Example 16, wherein edges of the first plate of high magnetic permeability material extend beyond edges of the at least one coil.

In Example 18, the catheter of either of Examples 16 or 17, wherein the at least one inductive sensor further includes a second plate of high magnetic permeability material, wherein the at least one coil is disposed between the first plate of high magnetic permeability material and the second plate of high magnetic permeability material.

In Example 19, the catheter of Example 18, wherein edges of the first plate of high magnetic permeability material and edges of the second plate of high magnetic permeability material extend beyond edges of the at least one coil.

In Example 20, the catheter of any of Examples 16-19, wherein the at least one coil is a flat coil of a flexible printed circuit.

In Example 21, the catheter of any of Examples 16-20, wherein the at least one coil includes a plurality of axially spaced coils.

In Example 22, the catheter of any of Examples 16-21, wherein the plurality of magnets consists of three magnets and the plurality of inductive sensor consists of three inductive sensors circumferentially arrayed evenly about a longitudinal axis.

In Example 23, the catheter of any of Examples 16-22, wherein the plurality of magnets are permanent magnets.

In Example 24, the catheter of any of Examples 16-23, wherein the plurality of magnets are disposed on the distal segment, and the plurality of inductive sensors are disposed on the proximal segment.

In Example 25, the catheter of any of Examples 16-24, wherein the distal segment includes an ablation element configured to deliver ablation therapy.

In Example 26, the catheter of Examples 16-25, wherein the signal indicative of the displacement between the inductive sensor and the opposite magnet is a change in an alternating voltage amplitude resulting from changes in a magnetic saturation of the first plate of high magnetic permeability material caused by changes in a distance between the inductive sensor and the corresponding opposite magnet.

Example 27 is a system adapted to measure a catheter contact force. The system includes a catheter and control circuitry. The catheter includes a proximal segment, a distal segment, a spring segment extending from the proximal segment to the distal segment, a plurality of magnets disposed on one of the proximal segment and the distal segment, and a plurality of inductive sensors disposed on the other one of the proximal segment and the distal segment. Each of the plurality of inductive sensors is opposite a different one of the plurality of magnets. The spring segment is configured to permit displacement between the distal segment and the proximal segment in response to an application of the force on the distal segment. Each of the plurality of inductive sensors includes a first plate of high magnetic permeability material and at least one coil disposed adjacent to the first plate of high magnetic permeability material. The coil is configured to output a signal indicative of the displacement between the inductive sensor and the corresponding opposite magnet. The control circuitry is configured to receive, for each of the plurality of inductive sensors, the signal indicative of the displacement between the magnet and the inductive sensor, and calculate at least one of a magnitude and a direction of the contact force based at least in part on the received signals.

In Example 28, the system of Example 27, wherein the at least one coil includes a plurality of axially spaced coils.

In Example 29, the system of either of Examples 27 or 28, wherein the spring segment includes an elastic element connecting the proximal segment to the distal segment to permit displacement between the distal segment and the proximal segment in response to an application of the force on the distal segment, and the control circuitry is further configured to calculate the at least one of the magnitude and the direction of the contact force based at least in part on a spring constant for the elastic element.

In Example 30, the system of any of Examples 27-29, wherein the plurality of magnets consists of three magnets and the plurality of inductive sensor consists of three inductive sensors circumferentially arrayed evenly about a longitudinal axis.

In Example 31, the system of any of Examples 27-30, wherein the at least one inductive sensor further includes a second plate of high magnetic permeability material, wherein the at least one coil is disposed between the first plate of high magnetic permeability material and the second plate of high magnetic permeability material.

In Example 32, the system of Example 31, wherein edges of the first plate of high magnetic permeability material and edges of the second plate of high magnetic permeability material extend beyond edges of the at least one coil.

In Example 33, the system of either of Examples 31 or 32, wherein the control circuitry is further configured to deliver an alternating sinusoidal electrical current to the at least one coil of each of the plurality of inductive sensors to produce an alternating voltage across the at least one coil, and wherein the signal indicative of the displacement between the inductive sensor and the corresponding opposite magnet is a change in an amplitude of the alternating voltage resulting from changes in a magnetic saturation of the first plate of high magnetic permeability material and the second plate of high magnetic permeability material caused by changes in a distance between the inductive sensor and the opposite magnet.

In Example 34, the system of any of Examples 27-33, wherein the distal segment includes an ablation element configured to deliver ablation therapy.

Example 35 is a method of determining a contact force exerted on a catheter having an elastic element disposed between a proximal segment having a plurality of coils disposed between plates of a high magnetic permeability material, and a distal segment having a plurality of magnets opposite the coils. The method includes delivering an alternating sinusoidal electrical current to the plurality of coils to produce an alternating voltage across each of the plurality of coils; measuring an amplitude of the alternating voltage produced across each of the plurality of coils, wherein for at least one of the plurality of coils, the amplitude of the alternating voltage decreases as the magnet opposite the coil moves closer to the coil and reduces an effective magnetic permeability of the plates of high magnetic permeability material as the contact force is exerted on the catheter; and calculating at least one of the magnitude and the direction of the contact force based on the measured amplitude of the alternating voltage produced across each of the plurality of coils and on a spring constant for the elastic element.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIGS. 1A-1C show a system for measuring a force with a catheter in accordance with various embodiments of this disclosure.

FIG. 2 shows a block diagram of circuitry for controlling various functions described herein.

FIG. 3 shows a perspective view of a distal end of a catheter in accordance with various embodiments of this disclosure.

FIG. 4 shows a side view of the inside of the catheter shown in FIG. 3 in accordance with various embodiments of this disclosure.

FIG. 5 shows a cross-sectional view of the catheter shown in FIG. 4 in accordance with various embodiments of this disclosure.

FIG. 6 shows a cross-sectional view of an inductive sensor in accordance with various embodiments of this disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Various cardiac abnormalities can be attributed to improper electrical activity of cardiac tissue. Such improper electrical activity can include, but is not limited to, generation of electrical signals, conduction of electrical signals, and/or mechanical contraction of the tissue in a manner that does not support efficient and/or effective cardiac function. For example, an area of cardiac tissue may become electrically active prematurely or otherwise out of synchrony during the cardiac cycle, thereby causing the cardiac cells of the area and/or adjacent areas to contract out of rhythm. The result is an abnormal cardiac contraction that is not timed for optimal cardiac output. In some cases, an area of cardiac tissue may provide a faulty electrical pathway (e.g., a short circuit) that causes an arrhythmia, such as atrial fibrillation or supraventricular tachycardia. In some cases, inactivate tissue (e.g., scar tissue) may be preferable to malfunctioning cardiac tissue.

Cardiac ablation is a procedure by which cardiac tissue is treated to inactivate the tissue. The tissue targeted for ablation may be associated with improper electrical activity, as described above. Cardiac ablation can lesion the tissue and prevent the tissue from improperly generating or conducting electrical signals. For example, a line, a circle, or other formation of lesioned cardiac tissue can block the propagation of errant electrical signals. In some cases, cardiac ablation is intended to cause the death of cardiac tissue and to have scar tissue reform over the lesion, where the scar tissue is not associated with the improper electrical activity. Lesioning therapies include electrical ablation, radiofrequency ablation, cyroablation, microwave ablation, laser ablation, and surgical ablation, among others. While cardiac ablation therapy is referenced herein as an exemplar, various embodiments of the present disclosure can be directed to ablation of other types of tissue and/or to non-ablation diagnostic and/or catheters that deliver other therapies.

Ideally, an ablation therapy can be delivered in a minimally invasive manner, such as with a catheter introduced into the heart through a vessel, rather than surgically opening the heart for direct access (e.g., as in a maze surgical procedure). For example, a single catheter can be used to perform an electrophysiology study of the inner surfaces of a heart to identify electrical activation patterns. From these patterns, a clinician can identify areas of inappropriate electrical activity and ablate cardiac tissue in a manner to kill or isolate the tissue associated with the inappropriate electrical activation. However, the lack of direct access in a catheter-based procedure may require that the clinician only interact with the cardiac tissue through a single catheter and keep track of all of the information that the catheter collects or is otherwise associated with the procedure. In particular, it can be challenging to determine the location of the therapy element (e.g., the proximity to tissue), the quality of a lesion, and whether the tissue is fully lesioned, under-lesioned (e.g., still capable of generating and/or conducting unwanted electrical signals), or over-lesioned (e.g., burning through or otherwise weakening the cardiac wall). The quality of the lesion can depend on the degree of contact between the ablation element and the targeted tissue. For example, an ablation element that is barely contacting tissue may not be adequately positioned to deliver effective ablation therapy. Conversely, an ablation element that is pressed too hard into tissue may deliver too much ablation energy or cause a perforation.

The present disclosure concerns, among other things, methods, devices, and systems for assessing a degree of contact between a part of a catheter (e.g., an ablation element) and tissue. Knowing the degree of contact, such as the magnitude and the direction of a force generated by contact between the catheter and the tissue, can be useful in determining the degree of lesioning of the targeted tissue. Information regarding the degree of lesioning of cardiac tissue can be used to determine whether the tissue should be further lesioned or whether the tissue was successfully ablated, among other things. Additionally or alternatively, an indicator of contact can be useful when navigating the catheter because a user may not feel a force being exerted on the catheter from tissue as the catheter is advanced within a patient, thereby causing vascular or cardiac tissue damage or perforation.

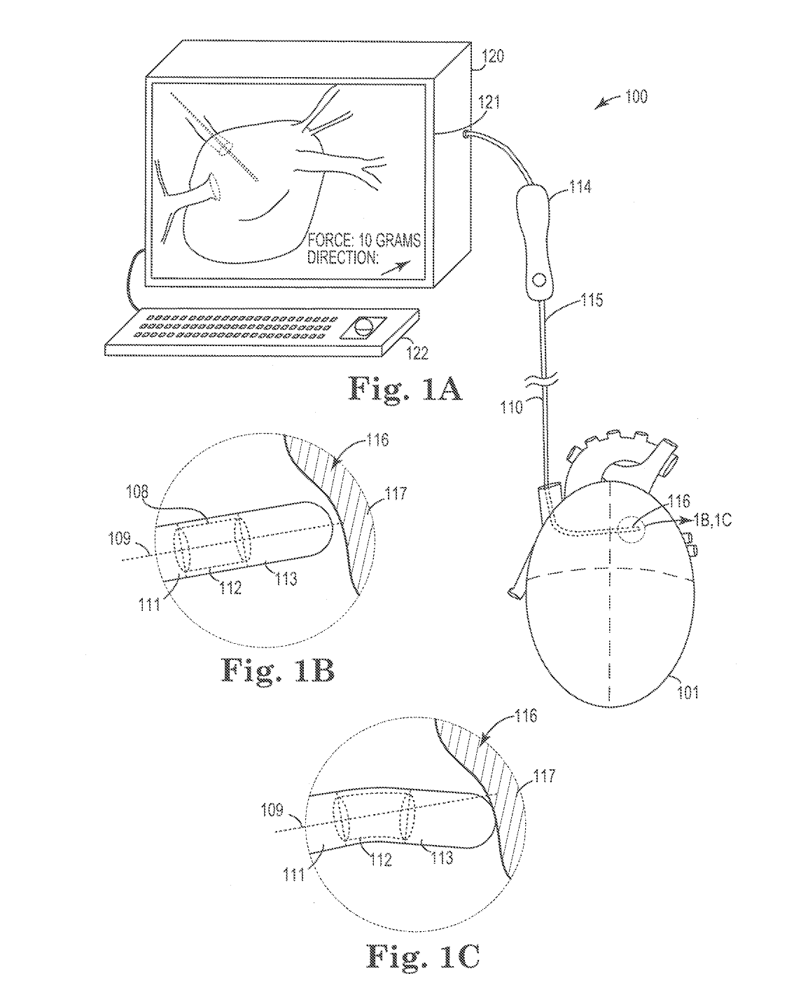

FIGS. 1A-1C illustrate an embodiment of a system 100 for sensing data from inside the body and/or delivering therapy. For example, the system 100 can be configured to map cardiac tissue and/or ablate the cardiac tissue, among other options. The system 100 includes a catheter 110 connected to a control unit 120 via handle 114. The catheter 110 can comprise an elongated tubular member having a proximal end 115 connected with the handle 114 and a distal end 116 configured to be introduced within a heart 101 or other area of the body. As shown in FIG. 1A, the distal end 116 of the catheter 110 is within the left atrium of the heart 101.

As shown in FIG. 1B, the distal end 116 of the catheter 110 includes a proximal segment 111, a spring segment 112, and a distal segment 113. The proximal segment 111, the spring segment 112, and the distal segment 113 can be coaxially aligned with each other in a base orientation as shown in FIG. 1B. Specifically, each of the proximal segment 111, the spring segment 112, and the distal segment 113 are coaxially aligned with a common longitudinal axis 109. The longitudinal axis 109 can extend through the radial center of each of the proximal segment 111, the spring segment 112, and the distal segment 113, and can extend through the radial center of the distal end 116 as a whole. The proximal segment 111, the spring segment 112, and the distal segment 113 can be mechanically biased to assume the base orientation. In some embodiments, the coaxial alignment of the proximal segment 111 with the distal segment 113 can correspond to the base orientation. As shown, the distal end 116, at least along the proximal segment 111, the spring segment 112, and the distal segment 113, extends straight. In some embodiments, this straight arrangement of the proximal segment 111, the spring segment 112, and the distal segment 113 can correspond to the base orientation.

The distal segment 113, or any other segment, can be in the form of an electrode configured for sensing electrical activity, such as electrical cardiac signals. In other embodiments, such an electrode can additionally or alternatively be used to deliver ablative energy to tissue.

The catheter 110 includes force sensing capabilities. For example, as shown in FIGS. 1B and 1C, the catheter 110 is configured to sense a force due to engagement with tissue 117 of heart 101. The distal segment 113 can be relatively rigid while segments proximal of the distal segment 113 can be relatively flexible. In particular, the spring segment 112 may be more flexible than the distal segment 113 and the proximal segment 111 such that when the distal end 116 of the catheter 110 engages tissue 117, the spring segment 112 bends, as shown in FIG. 1C. For example, the distal end 116 of the catheter 110 can be generally straight as shown in FIG. 1B. When the distal segment 113 engages tissue 117, the distal end 116 of the catheter 110 can bend at the spring segment 112 such that the distal segment 113 moves relative to the proximal segment 111. As shown in FIGS. 1B and 1C, the normal force from the tissue moves the distal segment 113 out of coaxial alignment (e.g., with respect to the longitudinal axis 109) with the proximal segment 111 while the spring segment 112 bends. As such, proximal segment 111 and the distal segment 113 may be stiff to not bend due to the force while the spring segment 112 may be less stiff and bend to accommodate the force exerted on the distal end 116 of the catheter 110. One or more sensors within the distal end 116 of the catheter 110 can sense the degree of bending or axial compression of the spring segment 112 to determine the magnitude and the direction of the force, as further discussed herein.

The control unit 120 of the system 100 includes a display 121 (e.g., a liquid crystal display or a cathode ray tube) for displaying information. The control unit 120 further includes a user input 122 which can include one or more buttons, toggles, a track ball, a mouse, touchpad, or the like for receiving user input. The user input 122 can additionally or alternatively be located on the handle 114. The control unit 120 can contain control circuitry for performing the functions referenced herein. Some or all of the control circuitry can alternatively be located within the handle 114.

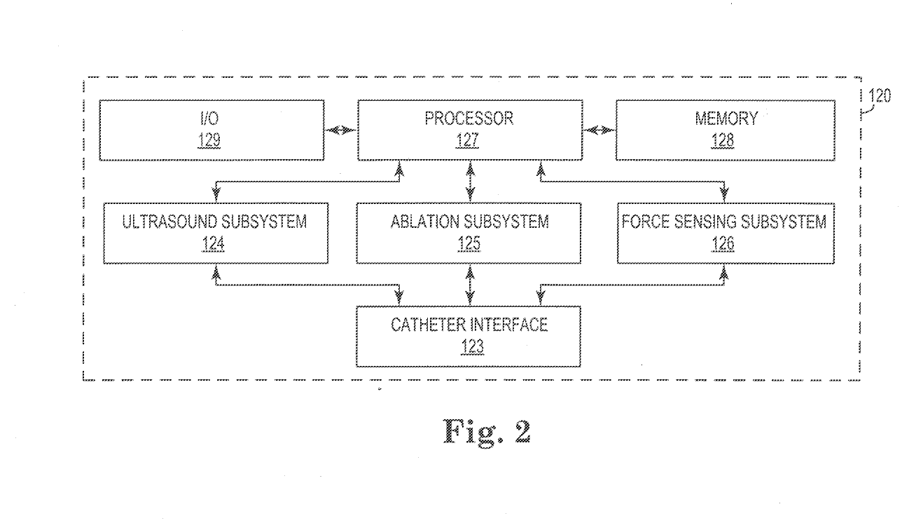

FIG. 2 illustrates a block diagram showing an example of control circuitry which can perform functions referenced herein. This or other control circuitry can be housed within control unit 120, which can comprise a single housing or multiple housings among which components are distributed. Control circuitry can additionally or alternatively be housed within the handle 114. The components of the control unit 120 can be powered by a power supply (not shown), as known in the art, which can supply electrical power to any of the components of the control unit 120 and the system 100. The power supply can plug into an electrical outlet and/or provide power from a battery, among other options.

The control unit 120 can include a catheter interface 123. The catheter interface 123 can include a plug which receives a cord from the handle 114. The catheter 110 can include multiple conductors (not illustrated but known in the art) to convey electrical signals between the distal end 116 and the proximal end 115 and further to the catheter interface 123. It is through the catheter interface 123 that the control unit 120 (and/or the handle 114 if control circuitry is included in the handle 114) can send electrical signals to any element within the catheter 110 and/or receive an electrical signal from any element within the catheter 110. The catheter interface 123 can conduct signals to any of the components of the control unit 120.

The control unit 120 can include an ultrasound subsystem 124 which includes components for operating the ultrasound functions of the system 100. While the illustrated example of control circuitry shown in FIG. 2 includes the ultrasound subsystem 124, it will be understood that not all embodiment may include ultrasound subsystem 124 or any circuitry for imaging tissue. The ultrasound subsystem 124 can include a signal generator configured to generate a signal for ultrasound transmission and signal processing components (e.g., a high pass filter) configured to filter and process reflected ultrasound signals as received by an ultrasound transducer in a sense mode and conducted to the ultrasound subsystem 124 through a conductor in the catheter 110. The ultrasound subsystem 124 can send signals to elements within the catheter 110 via the catheter interface 123 and/or receive signals from elements within the catheter 110 via the catheter interface 123.

The control unit 120 can include an ablation subsystem 125. The ablation subsystem 125 can include components for operating the ablation functions of the system 100. While the illustrated example of control circuitry shown in FIG. 2 includes the ablation subsystem, it will be understood that not all embodiment may include ablation subsystem 125 or any circuitry for generating an ablation therapy. The ablation subsystem 125 can include an ablation generator to provide different therapeutic outputs depending on the particular configuration (e.g., a high frequency alternating current signal in the case of radiofrequency ablation to be output through one or more electrodes). Providing ablation energy to target sites is further described, for example, in U.S. Pat. No. 5,383,874 and U.S. Pat. No. 7,720,420, each of which is expressly incorporated herein by reference in its entirety for all purposes. The ablation subsystem 125 may support any other type of ablation therapy, such as microwave ablation. The ablation subsystem 125 can deliver signals or other type of ablation energy through the catheter interface 123 to the catheter 110.

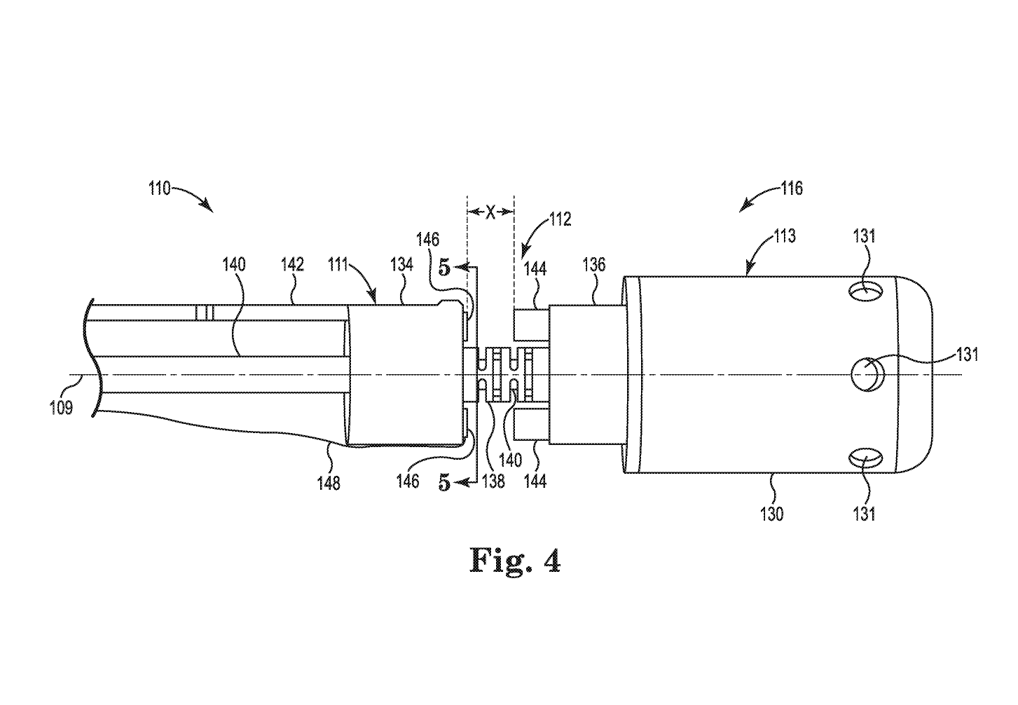

The control unit 120 can include a force sensing subsystem 126. The force sensing subsystem 126 can include components for measuring a force experienced by the catheter 110. Such components can include signal processors, analog-to-digital converters, operational amplifiers, comparators, and/or any other circuitry for conditioning and measuring one or more signals. The force sensing subsystem 126 can send electrical current to sensors, such as inductive sensors 146 (discussed below in reference to FIGS. 4-6), within the catheter 110 via the catheter interface 123 and receive signals from sensors within the catheter 110 via the catheter interface 123.

Each of the ultrasound subsystem 124, the ablation subsystem 125, and the force sensing subsystem 126 can send signals to, and receive signals from, the processor 127. The processor 127 can be any type of processor for executing computer functions. For example, the processor 127 can execute program instructions stored within the memory 128 to carry out any function referenced herein, such as determine the magnitude and direction of a force experienced by the catheter 110.

The control unit 120 further includes an input/output subsystem 129 which can support user input and output functionality. For example, the input/output subsystem 129 may support the display 121 to display any information referenced herein, such as a graphic representation of tissue, the catheter 110, and a magnitude and direction of the force experienced by the catheter 110, amongst other options. Input/output subsystem 129 can log key and/or other input entries via the user input 122 and route the entries to other circuitry.

A single processor 127, or multiple processors, can perform the functions of one or more subsystems, and as such the subsystems may share control circuitry. Although different subsystems are presented herein, circuitry may be divided between a greater or lesser numbers of subsystems, which may be housed separately or together. In various embodiments, circuitry is not distributed between subsystems, but rather is provided as a unified computing system. Whether distributed or unified, the components can be electrically connected to coordinate and share resources to carry out functions.

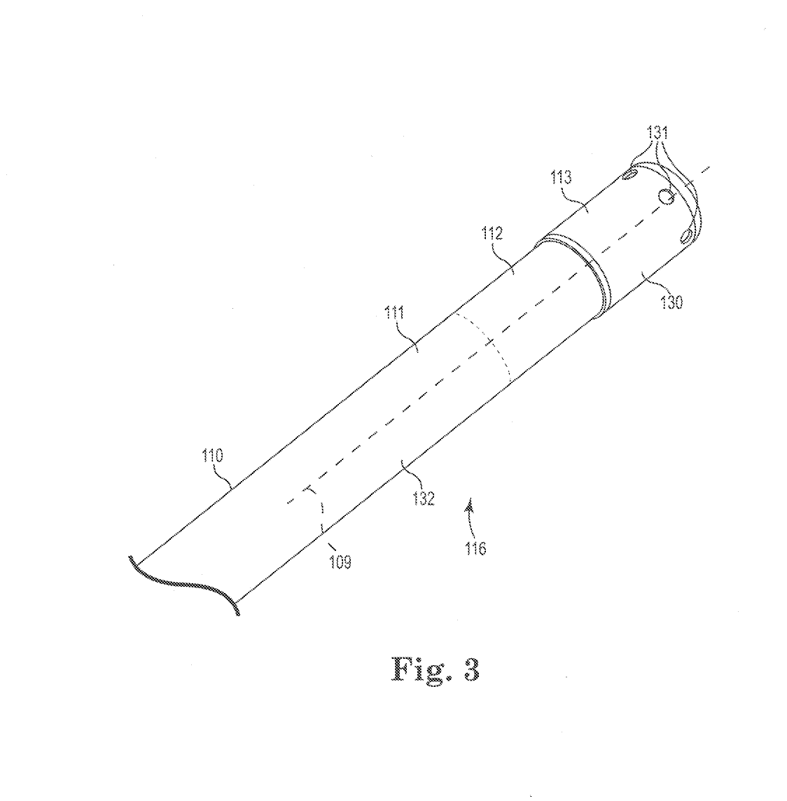

FIG. 3 illustrates a detailed view of the distal end 116 of the catheter 110. FIG. 3 shows a catheter shaft 132. The catheter shaft 132 can extend from the distal segment 113 to the handle 114 (FIG. 1A), and thus can define an exterior surface of the catheter 110 along the spring segment 112, the proximal segment 111, and further proximally to the proximal end 115 (FIG. 1A). The catheter shaft 132 can be a tube formed from various polymers, such as polyurethane, polyamide, polyether block amide, silicone, and/or other materials. In some embodiments, the catheter shaft 132 may be relatively flexible, and at least along the spring segment 112 may not provide any material mechanical support to the distal segment 113 (e.g., facilitated by thinning of the wall of the catheter shaft 132 along the spring segment 112).

As shown, the proximal segment 111 can be proximal and adjacent to the spring segment 112. The length of the proximal segment 111 can vary between different embodiments, and can be five millimeters to five centimeters, although different lengths are also possible. The length of the spring segment 112 can also vary between different embodiments and is dependent on the length of underlying features as will be further discussed herein. The spring segment 112 is adjacent to the distal segment 113. As shown in FIG. 3, the distal segment 113 can be defined by an electrode 130. The electrode 130 can be an ablation electrode. In some other embodiments, the distal segment 113 may not be an electrode. The electrode 130 can be in a shell form which can contain other components. The electrode 130 can include a plurality of ports 131. In some embodiments, the ports 131 may be fluidly connected to a source of irrigation fluid for flushing the volume adjacent to the distal segment 113. In some embodiments, one or more ultrasonic transducers, housed within the electrode 130, can transmit and receive signals through the ports 131 or through additional dedicated holes in the tip shell. Additionally or in place of the transducers, one or more miniature electrodes may be incorporated into the tip shell assembly.

FIG. 4 shows a side view of the inside of the distal end 116 of the catheter 110 of FIG. 3 after the removal of the catheter shaft 132 to expose various components that underlie the catheter shaft 132. As shown in FIG. 4, the proximal segment 111 may include a proximal hub 134, the distal segment 113 may include a distal hub 136, and the spring segment 112 may include an elastic element 138. In some embodiments, the proximal hub 134 and the distal hub 136 can be ring-like structures to which opposite ends of the elastic element 138 are attached to connect the proximal segment 111 to the distal segment 113. In other embodiments, the proximal hub 134, the distal hub 136, and the elastic element 138 may be integrally formed. One or both of the proximal hub 134 and the distal hub 136 can be formed from polymer materials, such as polyethylene, or PEEK, or can be formed from a metal, such as stainless steel. One or both of the proximal hub 134 and the distal hub 136 can be formed from a composite of metal, polymer, and/or other materials. The elastic element 138 provides predictable resistance to movement of the distal segment 113 relative to the proximal segment 111 according to a relationship governed by Hooke's law, in which force is a function of displacement and a spring constant. The elastic element 138 can be formed from a resilient material, for example, polymer materials, metals (e.g. stainless steel, nitinol), or other materials. In some embodiments, the elastic element 138 may be formed from a stainless steel hypotube.

The spring segment 112 can be a relatively flexible portion that is mostly or entirely mechanically supported by the elastic element 138. As such, the proximal hub 134 and the distal hub 136 can be stiffer than the elastic element 138 such that a force directed on the distal segment 113 causes the distal end 116 to bend along the elastic element 138 rather than along the distal segment 113 or the proximal segment 111.

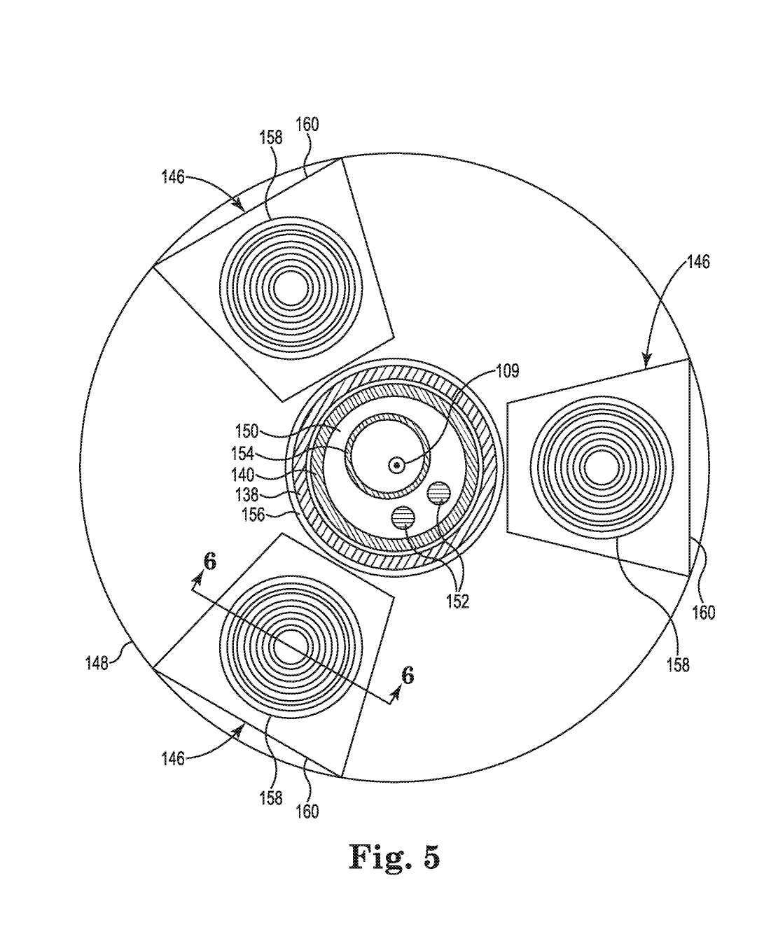

In the base orientation, the proximal hub 134, the distal hub 136, and the elastic element 138 can be coaxially aligned with respect to the longitudinal axis 109, as shown in FIG. 4. For example, the longitudinal axis 109 can extend through the respective radial centers of each of the proximal hub 134, the distal hub 136, and the elastic element 138. An inner tube 140, described below in reference to FIG. 5, can extend through the catheter 110 (e.g., from the handle 114, FIG. 1A), through the proximal hub 134, the elastic element 138, and the distal hub 136.

A tether 142 can attach to a proximal end of the proximal hub 134. Considering FIGS. 1A and 4, together, the tether 142 can attach to a deflection mechanism within the handle 114 to cause deflection of the distal end 116. A knob, slider, or plunger on a handle 114 may be used to create tension or slack in the tether 142.

As shown in FIG. 4, the distal end 116 of the catheter 110 further includes at least one magnet 144 and at least one inductive sensor 146. The at least one magnet 144 can be disposed on an axial-facing proximal surface of the distal hub 136 of the distal segment 113. The at least one inductive sensor 146 can be disposed on an axial-facing surface of the proximal hub 134 of the proximal segment 111 opposite the at least one magnet 144 such that the magnet 144 and the inductive sensor 146 are separated by a distance X. In some embodiments, such as the embodiment shown in FIG. 4, the at least one magnet 144 includes a plurality of magnets 144, and the at least one inductive sensor 146 includes a plurality of inductive sensors 146. In the embodiment shown in FIG. 4, the plurality of magnets 144 consists of three magnets 144 (two shown in FIG. 4) and the plurality of inductive sensors 146 consists of three inductive sensors 146 (two shown in FIG. 4). Each of the inductive sensors 146 is disposed opposite a corresponding different one of the magnets 144. The inductive sensors 146 can be electrically connected to the catheter interface 123 (FIG. 2) by way of a flexible printed circuit 148.

Although the embodiment shown in FIG. 4 shows the at least one magnet 144 disposed on the distal segment 113 and the inductive sensor 146 disposed the proximal segment 111 opposite the at least one magnet 144, it is understood that this disclosure encompasses embodiments in which the positions are reversed. That is, in some embodiments, at least one magnet 144 can be disposed on the proximal segment 111 and the inductive sensor 146 can be disposed the distal segment 113 opposite the at least one magnet 144.

The magnet 144 can generate a persistent, static magnetic field. In some embodiments, the magnet 144 may be one or more permanent magnets formed of a ferromagnetic material, such as cobalt, nickel, or iron, or combinations of metals or rare earth elements. In other embodiments, the magnet 144 can be an electromagnet energized by a static direct current. In some other embodiments, the magnet 144 can be a combination of one or more permanent magnets and an electromagnet.

In operation, when a contact force on the distal segment 113 causes the distal end 116 to bend along the elastic element 138, the distance X between a particular pair of the inductive sensor 146 and the opposite magnet 144 may change to varying degrees, depending on the location of the particular pair relative to the contact force. Each of the inductive sensors 146 outputs a signal indicative of the displacement between the inductive sensor 146 and its opposite magnet 144 as the distance X changes, as described below.

FIG. 5 shows a cross-sectional view of the distal end 116 of the catheter 110 shown in FIG. 4. As shown in FIG. 5, the inner tube 140 can include a lumen 150 within which one or more conductors 152 can extend from the proximal end 115 (FIG. 1A) to the distal segment 113, such as for connecting with one or more electrical elements (e.g., ultrasound transducer, electrode, or other component). Coolant fluid can additionally or alternatively be routed through the inner tube 140 by way of a coolant tube 154. In various embodiments, the catheter 110 is open irrigated (e.g., through the plurality of ports 131) to allow the coolant fluid to flow out of the distal segment 113. Various other embodiments concern a non-irrigated catheter 110. The flexible printed circuit 148 can be a physical substrate for the inductive sensors 146, in addition to electrically connecting them to the catheter interface 123 (FIG. 2). The flexible printed circuit 148 can include an opening 156 to accommodate the inner tube 140.

In the embodiment shown in FIG. 5, the distal end 116 of the catheter 110 includes three inductive sensors 146 at evenly spaced azimuth angles about the longitudinal axis 109 and at the same radial distance from the longitudinal axis 109. In other embodiments, the inductive sensors 146 may not be at evenly spaced azimuth angles and/or at the same radial distance from the longitudinal axis 109. In the embodiment shown in FIGS. 4 and 5, the inductive sensors 146 are in a coplanar configuration. In other embodiments, the inductive sensors 146 may not be in a coplanar configuration.

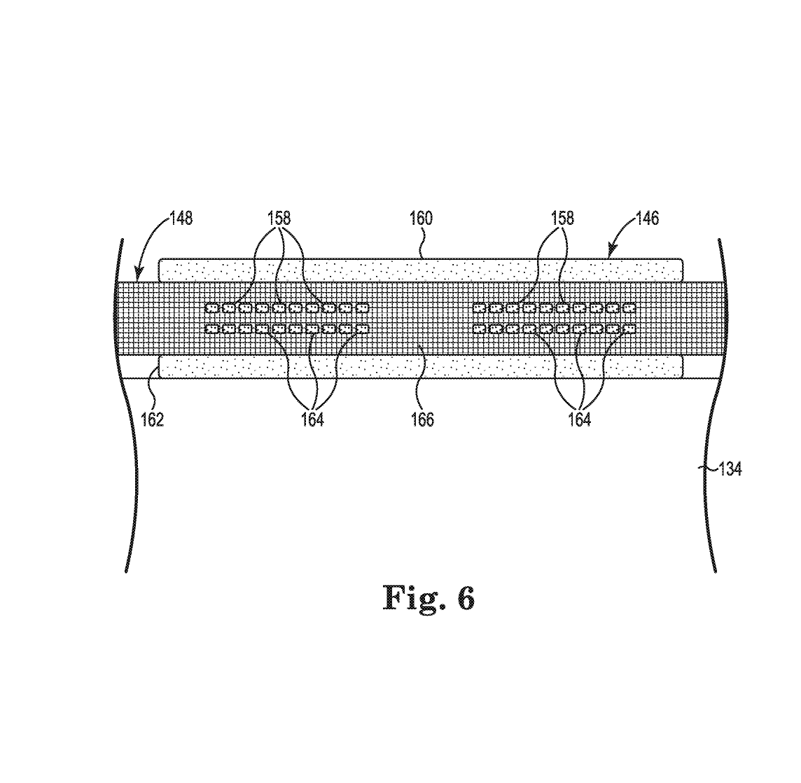

FIG. 6 shows a cross-sectional view of one of the inductive sensors 146. Considering FIGS. 5 and 6 together, each of the inductive sensors 146 includes at least one coil 158, a first plate 160, and a second plate 162. The embodiment of FIGS. 5 and 6 further includes another coil 164 such that the inductive sensor 146 includes a plurality of coils. The plurality of coils 158 and 164 can be electrically connected in series and are axially spaced and aligned to form a stack of coils, as shown in FIG. 6. Other embodiments may include more than two axially spaced coils. The coils 158 and 164 can be flat coils of a conductor, such as copper, gold, or combinations thereof, formed by concentric turns of conductive layers of the flexible printed circuit 148. In some embodiments, a thickness of the coils 158 and 164 can range from about 4 to about 10 microns and a width of individual turns can range from about 5 to about 10 microns. In other embodiments, the coils 158 and 164 can be physically separated from, but electrically connected to, the flexible printed circuit 148.

The first plate 160 and the second plate 162 can be plates, films, sheets, or coatings of a high magnetic permeability material, for example, a cobalt-based magnetic alloy. In some embodiments, the first plate 160 and the second plate 162 may each have a thickness ranging from about 10 microns to about 30 microns. In some embodiments, the first plate 160 and the second plate 162 each have a relative permeability of at least about 1000.

While the embodiment shown in FIG. 6 includes the first plate 160 and the second plate 162, other embodiments may include only one of either the first plate 160 or the second plate 162. As shown in FIG. 6, the coils 158 and 164 can be disposed between the first plate 160 and the second plate 162. The coils 158 and 164 can be insulated from each other and from the first plate 158 and the second plate 160 by an insulating material 166. In some embodiments, the coils 158 and 164 can be imbedded in the insulating material 166 as conductive layers of the flexible circuit 148, as shown in FIG. 6. In some embodiments, the thickness of the insulating material 166 between coils 158 and 164, and the first plate 158 and the second plate 164 can be between about 6 microns and about 12 microns. The insulating material 166 can be, for example, a flexible, insulating polymer, such as a polyimide. Edges of the first plate 160 and the second plate 162 can extend beyond edges of the coils 158 and 164, as shown in FIGS. 5 and 6, to magnetically encapsulate the coils 158 and 164.

While the embodiment shown in FIG. 6 includes the first plate 160 and the second plate 162, other embodiments may include only one of either the first plate 160 or the second plate 162. Inductive sensors incorporating such single-plate embodiments do not magnetically encapsulate the coils 158 and 164 and may not be as sensitive to indicating the displacement between the inductive sensor and the opposite magnet 144 as the embodiments including both the first plate 160 and the second plate 162.

For the sake of brevity, the operation of the catheter 110 is described below with respect to the coil 158 with the understanding that the description applies as well to coil 164 and any additional coils stacked between the first plate 160 and the second plate 162. In operation, the force sensing subsystem 126 (FIG. 2) supplies an alternating sinusoidal excitation current of frequency f and magnitude M to the coil 158 of each of the inductive sensors 146. The time-dependent excitation current M(t) may be described according to Equation 1:

M(t)=M sin(2πft). Eq. 1

The excitation current M(t) produces an alternating magnetic field of magnetic flux through and around the coil 158. The alternating magnetic field of magnetic flux is almost entirely contained within and between the first plate 160 and the second plate 162 because of their high magnetic permeability and because they extend beyond the edges of the coil 158 to effectively magnetically encapsulate the coil 158, as described above. By containing the alternating magnetic field of magnetic flux, the inductive sensors 146 are also less likely to generate external magnetic fields which could interfere with other systems, such as, for example, magnetic sensors used for navigation, as described below. The excitation current M(t) passing through the coil 158 develops a time dependent voltage having an amplitude V(t) across the coil 158. The voltage V(t) is a function of the magnitude of the time derivative of the excitation current M(t) and the inductance L of the coil 158. The voltage V(t) may be described according to Equation 2:

V(t)=L2πfM cos(2πft). Eq. 2

The inductance L of the coil 158 can be a function of a number of coil turns N, a cross-sectional coil area A, a coil length G, and the effective magnetic permeability μ(X) proximate to the coil 158. The inductance L may be described according to Equation 3:

L=N²Aμ(X)/G. Eq. 3

The number of turns N, the cross-sectional area A, and the length G are fixed parameters of the coil 158. The effective magnetic permeability μ(X), however, is a function of the permeability of the high magnetic permeability material of the first plate 160 and the second plate 162, and a distance X between the inductive sensor 146 and its corresponding opposite magnet 144. For example, as the distance X decreases in response to a force applied to the distal segment 113, bringing the magnet 144 closer to the inductive sensor 146, the static magnetic field of the magnet 144 interacts more strongly with the high permeability material of the first plate 160 and the second plate 162. This interaction reduces the effective permeability μ(X) of the first plate 160 and the second plate 162. Conversely, as the magnet 144 moves away from the inductive sensor 146, less of the magnetic flux of the magnet 144 interacts with the high permeability material of the first plate 160 and the second plate 162, increasing the effective permeability μ(X) of the first plate 160 and the second plate 162. In this way, the effective permeability μ(X) of the first plate 160 and the second plate 162 may be modulated by the force exerted on the distal segment 113 of the catheter 110 (FIGS. 1B and 1C). Without wishing to be bound by any theory, it is believed that as the static magnetic field of the magnet 144 enters the high permeability material of the first plate 160 and the second plate 162, it partially saturates the high magnetic permeability material of the first plate 160 and the second plate 162, reducing its capacity to conduct additional magnetic flux, thereby reducing the effective permeability μ(X) of the first plate 160 and the second plate 162. The change in effective permeability μ(X) results in a change in the inductance L of the coils 158 as describe in Equation 3 above.

In some embodiments, the static field of the magnet 144 interacts almost entirely with high magnetic permeability material of the first plate 160 and the second plate 162, and does not interact with the coil 158 to any significant extent because the first plate 160 and the second plate 162 essentially magnetically encapsulate the coil 158. Thus, the movement of the magnet 144 does not directly generate a significant voltage change in the coil 158.

Combining Equations 2 and 3, the relationship between the voltage amplitude V(t) across the coil 158 and the change in the distance X between the inductive sensor 146 and its opposite magnet 144 may be described according to Equation 4:

V(t)=2πfM cos(2πft)N²Aμ(ΔX)/G. Eq. 4

Thus, as shown in Equation 4, each of the inductive sensors 146 can output a change in the voltage amplitude V(t) resulting from a change in the magnetic saturation of the first plate 160 and the second plate 162 caused by changes in the distance X between the inductive sensor 146 and the corresponding opposite magnet 144.

As shown in FIG. 5, the three inductive sensors 146 are at evenly spaced azimuth angles about the longitudinal axis 109 and at the same radial distance from the longitudinal axis 109. If the force exerted on the distal segment 113 of the catheter 110 is coaxial with the longitudinal axis 109, then each of the inductive sensors 146 will output equal amounts of a change in the amplitude of the alternating voltage V(t) indicating an equal change in the distance X between each of the inductive sensors 146 and their corresponding opposite magnets 144. Based on these equal changes, the control circuitry can calculate a magnitude of the force exerted on the distal segment 113 based on the change in the distance X between each of the inductive sensors 146 and their corresponding opposite magnets 144, and the spring constant of the elastic element 138, according to Hooke's law. The control circuitry can also determine that the force is coaxial with the longitudinal axis 109 because the change in the amplitude of the alternating voltage V(t) is the same for each of the three inductive sensors 146.

If the force is not coaxial with the longitudinal axis 109, then distal segment 113 will tend to curl or shift radially away from the force with respect to the proximal segment 111. In such cases, the change in the amplitude of the alternating voltage V(t) for each of the inductive sensors 146 will not be equal. For example, in some cases, each of the inductive sensors 146 may output a different change in the amplitude of the alternating voltage V(t). In other cases, one or more inductive sensors 146 may output a different change in the amplitude of the alternating voltage V(t) compared to one or more other inductive sensors 146. Generally, the one or more inductive sensors 146 indicating the largest change in the amplitude of the alternating voltage V(t) indicate the opposite direction from which the force is coming. Based on this, the magnitude and the direction (e.g., unit vector) of the force can be determined by the control circuitry.

Once assembled, the catheter 110 may undergo a calibration step, either at a factory or just before use by a physician. In such a step, a plurality of forces of known magnitude and direction can be placed, in sequence, on the distal segment 113 to displace the distal segment 113 relative to the proximal segment 111, while the inductive sensors 146 output changes in the amplitude of their corresponding alternating voltage V(t). A mathematical relationship can be generated based on the linearity of Hooke's law, wherein a limited number of calibration steps are performed to determine the change in the amplitude of the alternating voltage V(t), and interpolation and/or extrapolation can be computed based on these calibration values. For example, the spring constant can be determined for the elastic element 138 such that subsequent changes in the distance X between the inductive sensor 146 and the opposite magnet 144 can be multiplied by the spring constant to determine the magnitude of the force acting on the distal segment 113. The changes in the distance X for multiple inductive sensors 146 can be factored for determining an overall magnitude and direction for the force.

The magnitude can be represented in grams or another measure of force. The magnitude can be presented as a running line graph, bar graph or graphic symbol varying with color or intensity that moves over time to show new and recent force values. The direction can be represented as a unit vector in a three dimensional reference frame (e.g., relative to an X, Y, and Z axes coordinate system). In some embodiments, a three dimensional mapping function can be used to track the three dimensional position of the distal end 116 of the catheter 110 in the three dimensional reference frame. Magnetic fields can be created outside of the patient and sensed by a sensor (not shown) that is sensitive to magnetic fields within distal end 116 of the catheter 110 to determine the three dimensional position and special orientation of the distal end 116 of the catheter 110 in the three dimensional reference frame. The direction can be represented relative to the distal end 116 of the catheter 110. For example, a line projecting to, or from, the distal segment 113 can represent the direction of the force relative to the distal segment 113. Similarly, a graphic symbol with varying color and/or intensity and/or shape could be utilized to represent the magnitude and/or the direction of the force. Such representations can be made on a display as discussed herein.

The magnitude and direction of the force can be used for navigation by providing an indicator when the catheter encounters tissue and/or for assessing the lesioning of tissue by determining the degree of contact between the lesioning element and the tissue, among other options. In some embodiments, a force under 10 grams is suboptimal for lesioning tissue (e.g., by being too small) while a force over 40 grams is likewise suboptimal for lesioning tissue (e.g., by being too large). Therefore, a window between 10 and 40 grams may be ideal for lesioning tissue and the output of the force during lesioning may provide feedback to the user to allow the user to stay within this window. Of course, other force ranges that are ideal for lesioning may be used.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A catheter adapted to measure a contact force, the catheter comprising:
a proximal segment;
a distal segment;
a spring segment extending from the proximal segment to the distal segment, the spring segment configured to permit displacement between the distal segment and the proximal segment in response to an application of the force on the distal segment;
a plurality of magnets disposed on one of the proximal segment and the distal segment; and
a plurality of inductive sensors disposed on the other one of the proximal segment and the distal segment, each of the plurality of inductive sensors opposite a different one of the plurality of magnets, each of the plurality of inductive sensors including:
a first plate of high magnetic permeability material; and
at least one coil disposed adjacent to the first plate of high magnetic permeability material, wherein the coil is configured to output a signal indicative of the displacement between the inductive sensor and the corresponding opposite magnet.

2. The catheter of claim 1, wherein edges of the first plate of high magnetic permeability material extend beyond edges of the at least one coil.

3. The catheter of claim 1, wherein the at least one inductive sensor further includes a second plate of high magnetic permeability material, wherein the at least one coil is disposed between the first plate of high magnetic permeability material and the second plate of high magnetic permeability material.

4. The catheter of claim 3, wherein edges of the first plate of high magnetic permeability material and edges of the second plate of high magnetic permeability material extend beyond edges of the at least one coil.

5. The catheter of claim 1, wherein the at least one coil is a flat coil of a flexible printed circuit.

6. The catheter of claim 1, wherein the at least one coil includes a plurality of axially spaced coils.

7. The catheter of claim 1, wherein the plurality of magnets consists of three magnets and the plurality of inductive sensor consists of three inductive sensors circumferentially arrayed evenly about a longitudinal axis.

8. The catheter of claim 1, wherein the plurality of magnets are permanent magnets.

9. The catheter of claim 1, wherein the plurality of magnets are disposed on the distal segment, and the plurality of inductive sensors are disposed on the proximal segment.

10. The catheter of claim 1, wherein the distal segment includes an ablation element configured to deliver ablation therapy.

11. The catheter of claim 1, wherein the signal indicative of the displacement between the inductive sensor and the opposite magnet is a change in an alternating voltage amplitude resulting from changes in a magnetic saturation of the first plate of high magnetic permeability material caused by changes in a distance between the inductive sensor and the corresponding opposite magnet.

12. A system adapted to measure a catheter contact force, the system comprising:
a catheter including:
a proximal segment;
a distal segment;
a spring segment extending from the proximal segment to the distal segment, the spring segment configured to permit displacement between the distal segment and the proximal segment in response to an application of the force on the distal segment;
a plurality of magnets disposed on the distal segment; and
a plurality of inductive sensors disposed on the proximal segment, each of the plurality of inductive sensors opposite a different one of the plurality of magnets, each of the plurality of inductive sensors including:
a first plate of high magnetic permeability material; and
at least one coil disposed between the first plate of high magnetic permeability material, wherein the coil is configured to output a signal indicative of the displacement between the inductive sensor and the opposite magnet; and
control circuitry configured to receive, for each of the plurality of inductive sensors, the signal indicative of the displacement between the magnet and the inductive sensor, and calculate at least one of a magnitude and a direction of the contact force based at least in part on the received signals.

13. The system of claim 12, wherein the at least one coil includes a plurality of axially spaced coils.

14. The system of claim 12, wherein the spring segment includes an elastic element connecting the proximal segment to the distal segment to permit displacement between the distal segment and the proximal segment in response to an application of the force on the distal segment, and the control circuitry is further configured to calculate the at least one of the magnitude and the direction of the contact force based at least in part on a spring constant for the elastic element.

15. The system of claim 12, wherein the plurality of magnets consists of three magnets and the plurality of inductive sensor consists of three inductive sensors circumferentially arrayed evenly about a longitudinal axis.

16. The system of claim 12, wherein the at least one inductive sensor further includes a second plate of high magnetic permeability material, wherein the at least one coil is disposed between the first plate of high magnetic permeability material and the second plate of high magnetic permeability material.

17. The system of claim 16, wherein edges of the first plate of high magnetic permeability material and edges of the second plate of high magnetic permeability material extend beyond edges of the at least one coil.

18. The system of claim 16, wherein the control circuitry is further configured to deliver an alternating sinusoidal electrical current to the at least one coil of each of the plurality of inductive sensors to produce an alternating voltage across the at least one coil, and wherein the signal indicative of the displacement between the inductive sensor and the corresponding opposite magnet is a change in an amplitude of the alternating voltage resulting from changes in a magnetic saturation of the first plate of high magnetic permeability material and the second plate of high magnetic permeability material caused by changes in a distance between the inductive sensor and the opposite magnet.

19. The system of claim 12, wherein the distal segment includes an ablation element configured to deliver ablation therapy.

20. A method of determining a contact force exerted on a catheter having an elastic element disposed between a proximal segment having a plurality of coils disposed between plates of a high magnetic permeability material, and a distal segment having a plurality of magnets opposite the coils, the method comprising:
delivering an alternating sinusoidal electrical current to the plurality of coils to produce an alternating voltage across each of the plurality of coils;
measuring an amplitude of the alternating voltage produced across each of the plurality of coils, wherein for at least one of the plurality of coils, the amplitude of the alternating voltage decreases as the magnet opposite the coil moves closer to the coil and reduces an effective magnetic permeability of the plates of high magnetic permeability material as the contact force is exerted on the catheter; and
calculating at least one of the magnitude and the direction of the contact force based on the measured amplitude of the alternating voltage produced across each of the plurality of coils and on a spring constant for the elastic element.
